(19) United States
(12) Patent Application Publication
(10) Pub. No.: US 2022/0151436 A1
(43) Pub. Date: May 19, 2022

(54) COOKING ASSEMBLIES AND METHODS OF OPERATION BASED ON A DETECTED COOKING OIL

(71) Applicant: Haier US Appliance Solutions, Inc., Wilmington, DE (US)

(72) Inventor: Ryan Newquist, Louisville, KY (US)

(21) Appl. No.: 17/099,173

(22) Filed: Nov. 16, 2020

(51) Int. Cl. A47J 37/12

(52) U.S. Cl. CPC A47J 37/1266; G01N 33/03; A47J 37/1209

(57) ABSTRACT

A cooking assembly or method of operating the same may include features for receiving an image signal from a camera assembly of a cooking zone; identifying a cooking oil based on the received image signal; and directing a heating element according to the identified cooking oil.

### FIELD OF THE INVENTION

The present subject matter relates generally to cooking assemblies and more particularly to systems for aiding and adjusting cooking operations of a cooking appliance.

### BACKGROUND OF THE INVENTION

Cooking appliances, such as cooktop or range appliances generally include heating elements for heating cookware item, such as pots, pans and griddles. A variety of configurations can be used for the heating elements located on the cooking surface of the cooktop. The number of heating elements or positions available for heating on the range appliance can include, for example, four, six, or more depending upon the intended application and preferences of the buyer. These heating elements can vary in size, location, and capability across the appliance.

Irrespective of the configuration of the cooking appliance itself, it is common for users of a cooking appliance to use various types of cooking oils (e.g., vegetable oil, canola oil, olive oil, flaxseed oil, coconut oil, butter, vegetable shortening, lard, margarine, etc.) when heating or preparing food items. Although certain types of cooking oils may be used interchangeably, the type of cooking oil selected by a user can have significant or subtle impacts in the cooking process. For instance, different cooking oils often have different smoke or ignition temperatures. Additionally or alternatively, different cooking oils may be more effective or flavorful when heated to different temperatures. In spite of these different characteristics, though, it is often difficult for a user to be aware of them. Moreover, it can be especially difficult for a user to know if a specific cooking oil is being used properly (e.g., cooked properly, maintained at an appropriate temperature, preventing from smoking, etc.).

As a result, there is a need for a cooking assembly or method that can adapt to various types of cooking oils. In particular, it would be advantageous if a cooking assembly could adapt to the type of cooking oil being used without requiring direct user input or knowledge of the characteristics the particular cooking oil being used.

### BRIEF DESCRIPTION OF THE INVENTION

Aspects and advantages of the invention will be set forth in part in the following description, or may be obvious from the description, or may be learned through practice of the invention.

In one exemplary aspect of the present disclosure, a cooking assembly is provided. The cooking assembly may include a cooking appliance, a camera assembly, and a controller. The cooking appliance may include a heating element defining a cooking zone selectively heated by the heating element. The camera assembly may be directed at the cooking zone to capture one or more images thereof. The controller in operable communication with the heating element and the camera assembly. The controller may be configured to initiate a cooking operation. The cooking operation may include receiving an image signal from a camera assembly adjacent to the cooking zone, identifying a cooking oil based on the received image signal, and directing the heating element according to the identified cooking oil.

In another exemplary aspect of the present disclosure, a method of operating a cooking appliance is provided. The method may include receiving an image signal from a camera assembly adjacent to a cooking zone; identifying a cooking oil based on the received image signal; and directing a heating element according to the identified cooking oil.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a front perspective view of a system according to exemplary embodiments of the present disclosure.

FIG. 2 provides a side schematic view of the exemplary system of FIG. 1.

FIG. 3 provides a bottom perspective view of a portion of the exemplary system of FIG. 1.

FIG. 4 provides a schematic view of a system for user engagement according to exemplary embodiments of the present disclosure.

FIG. 5 provides a flow chart illustrating a method of operating a system according to exemplary embodiments of the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the term “or” is generally intended to be inclusive (i.e., “A or B” is intended to mean “A or B or both”). The terms “first,” “second,” and “third” may be used interchangeably to distinguish one component from another and are not intended to signify location or importance of the individual components.

Generally, the present disclosure provides methods and systems for automatically identifying and responding to a type of cooking oil (e.g., vegetable oil, canola oil, olive oil, flaxseed oil, coconut oil, butter, vegetable shortening, lard, margarine, etc.) being used during a cooking operation.

As shown cooking appliance 300 defines a vertical direction V, a lateral direction L, and a transverse direction T, for example, at a cabinet 310. The vertical, lateral, and transverse directions are mutually perpendicular and form an orthogonal direction system. As shown, cooking appliance 300 extends along the vertical direction V between a top portion 312 and a bottom portion 314; along the lateral direction L between a left side portion and a right side portion; and along the traverse direction T between a front portion and a rear portion.

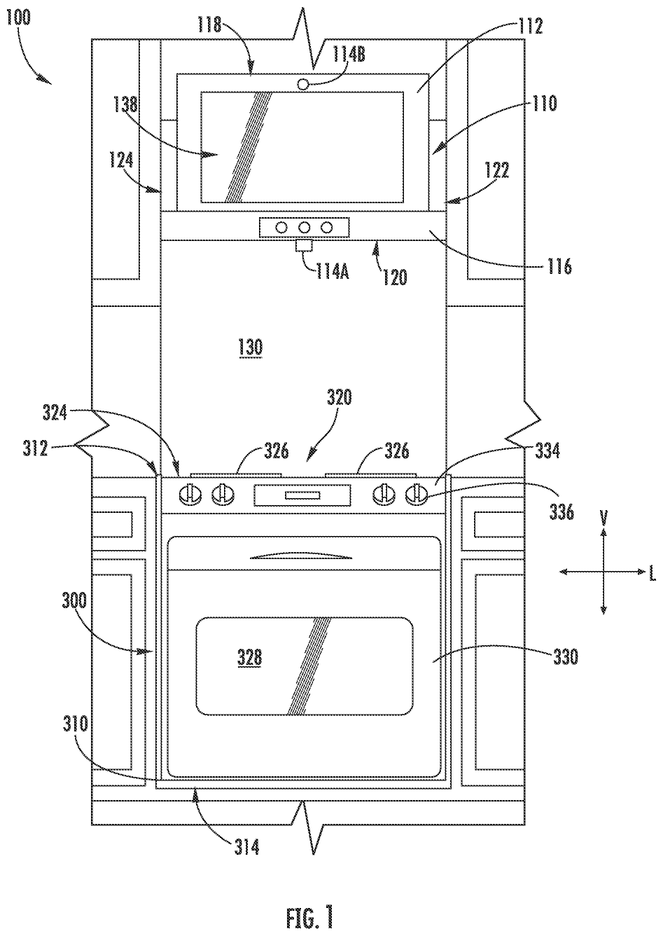

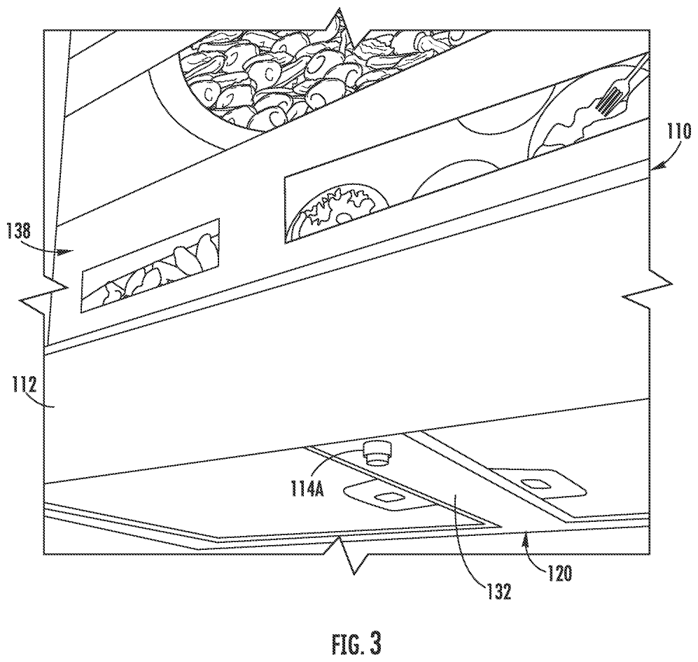

Turning to the figures, FIGS. 1 through 4 provide various views of a system 100 (or portions thereof) according to exemplary embodiments of the present disclosure. System 100 generally includes a stationary interactive assembly 110 with which a user may interact or engage. Interactive assembly 110 may have a controller 510A that is in operable communication with an image monitor 112 and one or more camera assemblies (e.g., camera assembly 114A and camera assembly 114B) that are generally positioned above a cooking appliance 300.

Cooking appliance 300 can include a chassis or cabinet 310 that defines a cooking zone 320 wherein one or more cooking operations may be performed by a user (e.g., heating or preparing food items according to a recipe). For example, the cooking zone 320 may be defined by a cooktop surface 324 of the cabinet 310. As illustrated, cooktop surface 324 includes one or more heating elements 326 for use in, for example, heating or cooking operations. In exemplary embodiments, cooktop surface 324 is constructed with ceramic glass. In other embodiments, however, cooktop surface 324 may include another suitable material, such as a metallic material (e.g., steel) or another suitable non-metallic material. Heating elements 326 may be various sizes and may employ any suitable method for heating or cooking an object, such as a cookware item 338, and its contents. In one embodiment, for example, heating element 326 uses a heat transfer method, such as electric coils or gas burners, to heat the cookware item 338. In another embodiment, however, heating element 326 uses an induction heating method to heat the cookware item 338 directly. In turn, heating element 326 may include a gas burner element, resistive heat element, radiant heat element, induction element, or another suitable heating element.

In some embodiments, cooking appliance 300 includes an insulated cabinet 310 that defines a cooking chamber 328 selectively covered by a door 330. One or more heating elements 332 (e.g., top broiling elements or bottom baking elements) may be enclosed within cabinet 310 to heat cooking chamber 328. Heating elements 332 within cooking chamber 328 may be provided as any suitable element for cooking the contents of cooking chamber 328, such as an electric resistive heating element, a gas burner, microwave element, halogen element, etc. Thus, cooking appliance 300 may be referred to as an oven range appliance. As will be understood by those skilled in the art, cooking appliance 300 is provided by way of example only, and the present subject matter may be used in any suitable cooking appliance 300, such as a double oven range appliance, standalone oven, or a standalone cooktop (e.g., fitted integrally with a surface of a kitchen counter). Thus, the example embodiments illustrated in figures are not intended to limit the present subject matter to any particular cooking chamber or heating element configuration, except as otherwise indicated.

As illustrated, a user interface or user interface panel 334 may be provided on cooking appliance 300. Although shown at the front portion of cooking appliance 300, another suitable location or structure (e.g., a backsplash) for supporting user interface panel 334 may be provided in alternative embodiments. In some embodiments, user interface panel 334 includes input components or controls 336, such as one or more of a variety of electrical, mechanical, or electro-mechanical input devices. Controls 336 may include, for example, rotary dials, knobs, push buttons, and touch pads. A controller 510C is in communication with user interface panel 334 and controls 336 through which a user may select various operational features and modes and monitor progress of cooking appliance 300. In additional or alternative embodiments, user interface panel 334 includes a display component, such as a digital or analog display in communication with a controller 510C and configured to provide operational feedback to a user. In certain embodiments, user interface panel 334 represents a general purpose I/O (“GPIO”) device or functional block.

As shown, controller 510C is communicatively coupled (i.e., in operative communication) with user interface panel 334 and its controls 336. Controller 510C may also be communicatively coupled with various operational components of cooking appliance 300 as well, such as heating elements (e.g., 326, 332), sensors, etc. Input/output (“I/O”) signals may be routed between controller 510C and the various operational components of cooking appliance 300. Thus, controller 510C can selectively activate and operate these various components. Various components of cooking appliance 300 are communicatively coupled with controller 510C via one or more communication lines such as, for example, conductive signal lines, shared communication busses, or wireless communications bands.

In some embodiments, controller 510C includes one or more memory devices 514C and one or more processors 512C. The processors 512C can be any combination of general or special purpose processors, CPUs, or the like that can execute programming instructions or control code associated with operation of cooking appliance 300. The memory devices 514C (i.e., memory) may represent random access memory such as DRAM or read only memory such as ROM or FLASH. In one embodiment, the processor 512C executes programming instructions stored in memory 514C. The memory 514C may be a separate component from the processor 512C or may be included onboard within the processor 512C. Alternatively, controller 510C may be constructed without using a processor, for example, using a combination of discrete analog or digital logic circuitry (such as switches, amplifiers, integrators, comparators, flip-flops, AND gates, and the like) to perform control functionality instead of relying upon software.

In certain embodiments, controller 510C includes a network interface 520C such that controller 510C can connect to and communicate over one or more networks (e.g., network 502) with one or more network nodes. Controller 510C can also include one or more transmitting, receiving, or transceiving components for transmitting/receiving communications with other devices communicatively coupled with cooking appliance 300. Additionally or alternatively, one or more transmitting, receiving, or transceiving components can be located off board controller 510C. Generally, controller 510C can be positioned in any suitable location throughout cooking appliance 300. For example, controller 510C may be located proximate user interface panel 334 toward the front portion of cooking appliance 300.

As shown, one or more casings (e.g., hood casing 116) may be provided above cooking appliance 300 along the vertical direction V. For example, a hood casing 116 may be positioned above cooking appliance 300 in a stationary mounting (e.g., such that operation of interactive assembly 110 is not permitted unless casing 116 is mounted at a generally fixed or non-moving location). Hood casing 116 includes a plurality of outer walls and generally extends along the vertical direction V between a top end 118 and a bottom end 120; along the lateral direction L between a first side end 122 and a second side end 124; and along the transverse direction T between a front end 126 and a rear end 128. In some embodiments, hood casing 116 is spaced apart from cooking zone 320 or cooktop surface 324 along the vertical direction V. An open region 130 may thus be defined along the vertical direction V between cooking zone 320 or cooktop surface 324 and bottom end 120.

In optional embodiments, hood casing 116 is formed as a range hood. A ventilation assembly within hood casing 116 may thus direct an airflow from the open region 130 and through hood casing 116. However, a range hood is provided by way of example only. Other configurations may be used within the spirit and scope of the present disclosure. For example, hood casing 116 could be part of a microwave or other appliance designed to be located above cooking appliance 300 (e.g., directly above cooktop surface 324). Moreover, although a generally rectangular shape is illustrated, any suitable shape or style may be adapted to form the structure of hood casing 116.

In certain embodiments, one or more camera assemblies 114A, 114B are provided to capture images (e.g., static images or dynamic video) of a portion of cooking appliance 300 or an area adjacent to cooking appliance 300. Generally, each camera assembly 114A, 114B may be any type of device suitable for capturing a picture or video. As an example, each camera assembly 114A, 114B may be a video camera or a digital camera with an electronic image sensor [e.g., a charge coupled device (CCD) or a CMOS sensor]. Moreover, as is understood, each camera assembly 114A, 114B may include an infrared (IR) sensor 198A, 198B, (e.g., as a separate element or included within the electronic image sensor). A camera assembly 114A or 114B is generally provided in operable communication with controller 510A such that controller 510A may receive an image signal (e.g., video signal) from camera assembly 114A or 114B corresponding to the picture(s) captured by camera assembly 114A or 114B. Once received by controller 510A, the image signal (e.g., video signal) may be further processed at controller 510A (e.g., for viewing at image monitor 112) or transmitted to a separate device (e.g., remote server 404) “live” or in real-time for remote viewing (e.g., via one or more social media platforms). Optionally, one or more microphones (not pictured) may be associated with one or more of the camera assemblies 114A, 114B to capture and transmit audio signal(s) coinciding (or otherwise corresponding) with the captured image signal or picture(s).

In some embodiments, one camera assembly (e.g., first camera assembly 114A) is directed at cooking zone 320 (e.g., cooktop surface 324). In other words, first camera assembly 114A is oriented to capture light emitted or reflected from cooking zone 320 through the open region 130. In some such embodiments, first camera assembly 114A can selectively capture an image covering all or some of cooktop surface 324. For instance, first camera assembly 114A may capture an image covering one or more heating elements 326 of cooking appliance 300. In some such embodiments, the captured heating elements 326 and any cookware item 338 or object placed on or adjacent to one of the heating elements 326 (e.g., between cooking zone 320 and first camera assembly 114A) may be recorded and transmitted to another portion of system (e.g., image monitor 112), such as part of a real-time video feed. Thus, a video feed may include a digital picture or representation of the heating elements 326, cookware item 338 (e.g., including a volume of cooking oil 340 held therein), or object (e.g., oil storage vessel 342) in the line of sight of the video feed. Optionally, first camera assembly 114A may be directed such that a line of sight is defined from first camera assembly 114A that is perpendicular to cooktop surface 324.

As shown, first camera assembly 114A is positioned above cooking zone 320 (e.g., along the vertical direction V). In some such embodiments, first camera assembly 114A is mounted (e.g., fixedly or removably) to hood casing 116. A cross-brace 132 extending across hood casing 116 (e.g., along the transverse direction T) may support first camera assembly 114A. When assembled, first camera assembly 114A may be positioned directly above cooking zone 320 or cooktop surface 324.

In optional embodiments, one camera assembly (e.g., second camera assembly 114B) is directed away from cooking zone 320 or cooktop surface 324. In other words, second camera assembly 114B is oriented to capture light emitted or reflected from an area other than cooktop surface 324. In particular, second camera assembly 114B may be directed at the area in front of cooking appliance 300 (e.g., directly forward from cooking appliance 300 along the transverse direction T). Thus, second camera assembly 114B may selectively capture an image of the area in front of cooking zone 320. This area may correspond to or cover the location where a user would typically stand during use of cooking appliance 300. During use, a user's face or body may be captured by second camera assembly 114B while the user is standing directly in front of cooking appliance 300. Optionally, second camera assembly 114B may be directed such that a line of sight is defined from second camera assembly 114B that is non-orthogonal to cooktop surface 324 (e.g., between 0° and 45° relative to a plane parallel to cooktop surface 324). The captured images from second camera assembly 114B may be suitable for transmission to a remote device or may be processed as part of one or more operations of interactive assembly 110, such as a gesture control signal for a portion of interactive assembly 110 (e.g., to engage a graphical user interface displayed at image monitor 112) or identification of a cooking oil storage vessel.

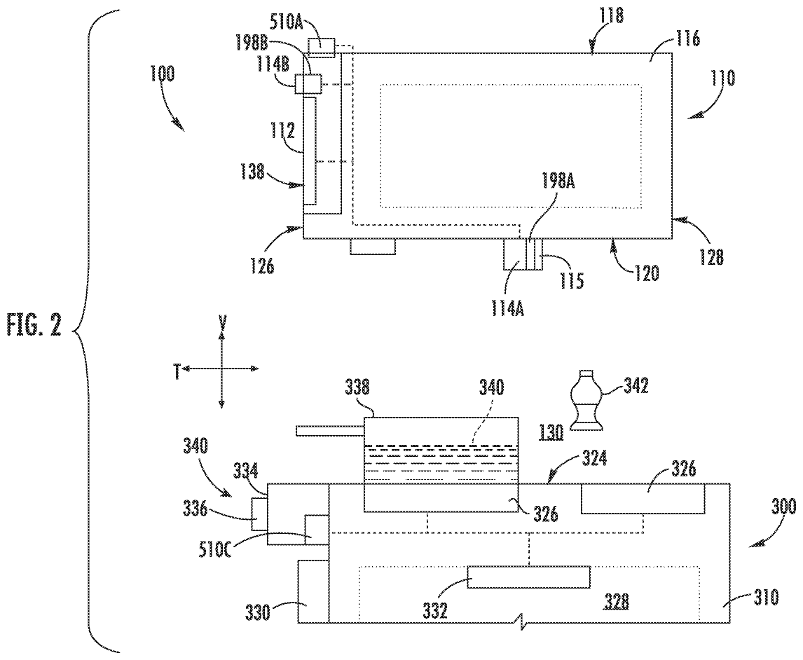

As shown, second camera assembly 114B is positioned above cooking appliance 300 (e.g., along the vertical direction V). In some such embodiments, such as that illustrated in FIGS. 1 and 2, second camera assembly 114B is mounted (e.g., fixedly or removably) to a front portion of hood casing 116 (e.g., at image monitor 112). When assembled, second camera assembly 114B may be positioned directly above a portion of cooking appliance 300 (e.g., cooking zone 320 or cooktop surface 324) or, additionally, forward from cooking appliance 300 along the transverse direction T.

During use of one or more of the camera assemblies 114A, 114A, such as during an image capture sequence, camera 114A or 114B may capture one or more two-dimensional images (e.g., as a video feed or series of sequential static images) that may be transmitted to the controller 510A (e.g., as a data or image signal), as is generally understood. From the captured images, a cooking oil (e.g., vegetable oil, canola oil, olive oil, flaxseed oil, coconut oil, butter, vegetable shortening, lard, margarine, etc.) within the field of view for the camera 114A or 114B may be automatically detected or identified by the controller 510A. As would be understood, detecting or identifying such items, may be performed by edge matching, divide-and-conquer search, greyscale matching, histograms of receptive field responses, or another suitable routine (e.g., executed at the controller 510A based on one or more captured images from camera 114A or 114B).

As an example, the cooking oil may be detected and identified as an applied or loose liquid volume 340 of oil within a cookware item 338 (i.e., outside of any storage vessel). The liquid volume 340 may be analyzed for color, reflectivity, or other visible characteristics. Additionally or alternatively, the presence smoke (e.g., image distortion caused thereby) may be detected from the liquid volume 340. The presence of smoke with, for instance, an identified temperature (e.g., detected at a discrete temperature sensor or the camera assembly 114A or 114A, such as by the IR sensor 198A or 198B) may indicate a smoke point or temperature (i.e., flash point, burning point, or temperature at which the oil begins to smoke or oxidize) that corresponds to a specific type of cooking oil.

As an additional or alternative example, the cooking oil may be detected an identified from a container or storage vessel 342 within which the oil is held. The storage vessel 342 may be analyzed for shape, color, or other visible characteristics. Additionally or alternatively, a container label affixed to or printed on the storage vessel 342 may be analyzed. For instance, the container label may be scanned by the camera assembly 114A or 114B and one or more character recognition sequences may be initiated, as would be understood, in order to interpret text, images, or barcode markers included on the container label.

In some embodiments, a lighting assembly 134 is provided above cooktop surface 324 (e.g., along the vertical direction V). For instance, lighting assembly 134 may be mounted to hood casing 116 (e.g., directly above cooking zone 320 or cooktop surface 324). Generally, lighting assembly 134 includes one or more selectable light sources directed toward cooking zone 320. In other words, lighting assembly 134 is oriented to project a light (as indicated at arrows 136) to cooking appliance 300 through open region 130 and illuminate at least a portion of cooking zone 320 (e.g., cooktop surface 324). The light sources may include any suitable light-emitting elements, such as one or more light emitting diode (LED), incandescent bulb, fluorescent bulb, halogen bulb, etc.

During use, lighting assembly 134 may be selectively activated to illuminate a portion of cooking appliance 300 (e.g., cooktop surface 324) based on a received light visibility signal. For instance, lighting assembly 134 may be activated by controller 510A based on direct user input (e.g., depressing a dedicated switch, a gesture control signal, a voice control signal, etc.). In other words, the light visibility signal may be an isolated user input signal.

Alternatively, the light visibility signal may be an automatically-generated signal that does not require direct user input. As an example, lighting assembly 134 may be activated by controller 510A with or as part of the above-described image capture sequence (e.g., to ensure consistency of lighting or otherwise improve detection and identification of a cookware item 338 from a captured image). Thus, the light visibility signal may be transmitted in tandem with (e.g., in response to the same condition or signal) as the image capture signal. As an additional or alternative example, the light visibility signal may indicate additional light is needed above cooking appliance 300. In turn, controller 510A may automatically activate lighting assembly 134 based on a determined condition. Optionally, controller 510A may vary the activation or light intensity (i.e., luminance) of the light 136 from lighting assembly 134 based on the ambient conditions (e.g., through the open region 130 between cooking zone 320 and hood casing 116). For instance, an ambient light sensor 115 may be positioned above cooking zone 320 (e.g., directly above cooktop surface 324). In some such embodiments, ambient light sensor 115 detects the light available at first camera assembly 114A and transmits a corresponding light visibility signal to controller 510A. Based on the received light visibility signal, controller 510A may direct lighting assembly 134 to activate/deactivate or increase/decrease the intensity of light 136 projected towards cooking appliance 300.

In some embodiments, an image monitor 112 is provided, for instance, above top surface 324 (e.g., along the vertical direction V). In exemplary embodiments, image monitor 112 is mounted to hood casing 116 (e.g., directly above top surface 324). Generally, image monitor 112 may be any suitable type of mechanism for visually presenting an interactive or non-static image. For example, image monitor 112 may be a liquid crystal display (LCD), a plasma display panel (PDP), a cathode ray tube (CRT) display, etc. Thus, image monitor 112 includes an imaging surface 138 (e.g., screen or display panel) at which the digital image is presented or displayed as an optically-viewable picture (e.g., static image or dynamic video) to a user. The optically-viewable picture may correspond to any suitable signal or data received or stored by interactive assembly 110 (e.g., at controller 510A). As an example, image monitor 112 may present recipe information in the form of viewable text or images. As another example, image monitor 112 may present a remotely captured image, such as a live (e.g., real-time) dynamic video stream received from a separate user or device. As yet another example, image monitor 112 may present a graphical user interface (GUI) that allows a user to select or manipulate various operational features of interactive assembly 110 or cooking appliance 300. During use of such GUI embodiments, a user may engage, select, or adjust the image presented at image monitor 112 through any suitable input, such as gesture controls detected through second camera assembly 114B, voice controls detected through one or more microphones, associated touch panels (e.g., capacitance or resistance touch panel) or sensors overlaid across imaging surface 138, etc.

In certain embodiments, the imaging surface 138 is directed away from, top surface 324. In particular, the imaging surface 138 may be directed toward the area forward from the cooking appliance 300. During use, a user standing in front of cooking appliance 300 may thus see the optically-viewable picture (e.g., recipe, dynamic video stream, graphical user interface, etc.) displayed at the imaging surface 138. Optionally, the imaging surface 138 may be positioned at a rearward non-orthogonal angle relative to the vertical direction. In other words, the imaging surface 138 may be inclined such that an upper edge of the imaging surface 138 is closer to the rear end 128 of hood casing 116 than a lower edge of the imaging surface 138 is. In some such embodiments, the non-orthogonal angle is between 1° and 15° relative to the vertical direction V. In certain embodiments, the non-orthogonal angle is between 2° and 7° relative to the vertical direction V.

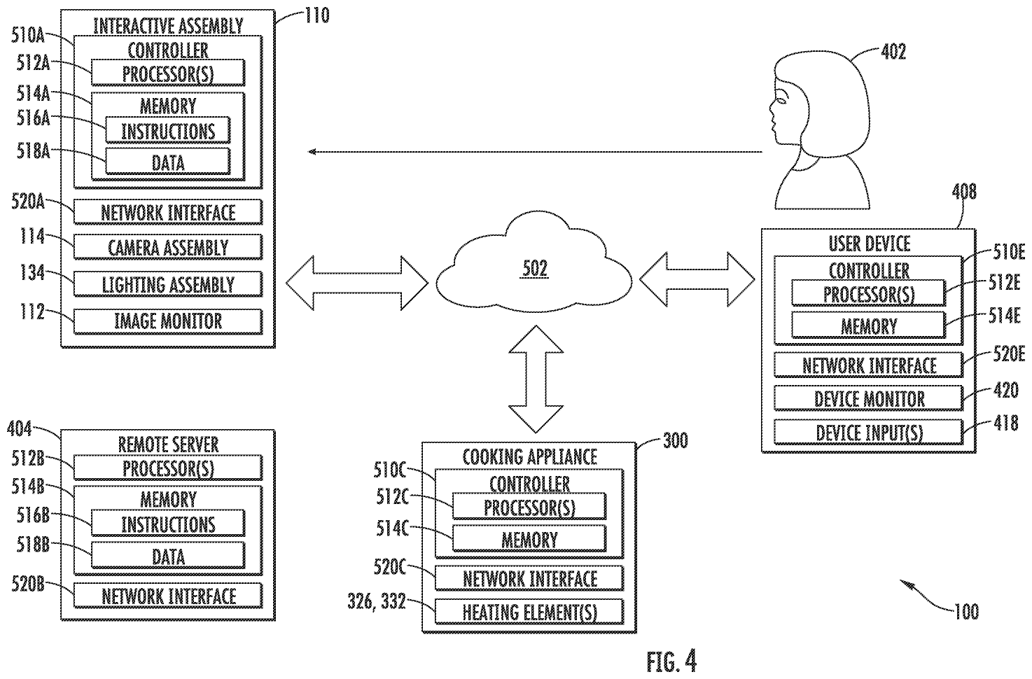

FIG. 4 provides a schematic view of a system 100 for user engagement according to exemplary embodiments of the present disclosure. As shown, interactive assembly 110 can be communicatively coupled with network 502 and various other nodes, such as a remote server 404, cooking appliance 300, and one or more user devices 408. Moreover, one or more users 402 can be in operative communication with interactive assembly 110 by various methods, including voice control or gesture recognition, for example. Additionally, or alternatively, although network 502 is shown, one or more portions of the system (e.g., interactive assembly 110, cooking appliance 300, user device 408, or other devices within system) may be communicatively coupled without network 502; rather, interactive assembly 110 and various other devices of the system can be communicatively coupled via any suitable wired or wireless means not over network 502, such as, for example, via physical wires, transceiving, transmitting, or receiving components.

As noted above, interactive assembly 110 may include a controller 510A operably coupled to one or more camera assemblies 114, lighting assemblies 134, and image monitors 110. Controller 510A may include one or more processors 512A and one or more memory devices 514A (i.e., memory). The one or more processors 512A can be any suitable processing device (e.g., a processor core, a microprocessor, an ASIC, a FPGA, a microcontroller, etc.) and can be one processor or a plurality of processors that are operatively connected. The memory device 514A can include one or more non-transitory computer-readable storage mediums, such as RAM, ROM, EEPROM, EPROM, flash memory device, magnetic disks, etc., and combinations thereof. The memory devices 514A can store data 518A and instructions 516A that are executed by the processor 512A to cause interactive assembly 110 to perform operations. For example, instructions 516A could be instructions for voice recognition, instructions for gesture recognition, receiving/transmitting images or image signals from camera assembly 114, directing activation of lighting assembly 134, or projecting images at image monitor 112. The memory devices 514A may also include data 518A, such as captured image data, notification or message data, etc., that can be retrieved, manipulated, created, or stored by processor 512A.

Controller 510A includes a network interface 520A such that interactive assembly 110 can connect to and communicate over one or more networks (e.g., network 502) with one or more network nodes. Network interface 520A can be an onboard component of controller 510A or it can be a separate, off board component. Controller 510A can also include one or more transmitting, receiving, or transceiving components for transmitting/receiving communications with other devices communicatively coupled with interactive assembly 110. Additionally or alternatively, one or more transmitting, receiving, or transceiving components can be located off board controller 510A.

Network 502 can be any suitable type of network, such as a local area network (e.g., intranet), wide area network (e.g., internet), low power wireless networks [e.g., Bluetooth Low Energy (BLE)], or some combination thereof and can include any number of wired or wireless links. In general, communication over network 502 can be carried via any type of wired or wireless connection, using a wide variety of communication protocols (e.g., TCP/IP, HTTP, SMTP, FTP), encodings or formats (e.g., HTML, XML), or protection schemes (e.g., VPN, secure HTTP, SSL).

In some embodiments, a remote server 404, such as a web server, is in operable communication with interactive assembly 110. The server 404 can be used to host an information database. The server can be implemented using any suitable computing device(s). The server 404 may include one or more processors 512B and one or more memory devices 514B (i.e., memory). The one or more processors 512B can be any suitable processing device (e.g., a processor core, a microprocessor, an ASIC, a FPGA, a microcontroller, etc.) and can be one processor or a plurality of processors that are operatively connected. The memory device 512B can include one or more non-transitory computer-readable storage mediums, such as RAM, ROM, EEPROM, EPROM, flash memory devices, magnetic disks, etc., and combinations thereof. The memory devices 514B can store data 518B and instructions 516B which are executed by the processor 512B to cause remote server 404 to perform operations. For example, instructions 516B could be instructions for receiving/transmitting images or image signals, transmitting/receiving oil characteristic signals, etc.

The memory devices 514B may also include data 518B, such as oil characteristic data, notification data, message data, image data, etc., that can be retrieved, manipulated, created, or stored by processor 512B. The data 518B can be stored in one or more databases. The one or more databases can be connected to remote server 404 by a high bandwidth LAN or WAN, or can also be connected to remote server 404 through network 502. The one or more databases can be split up so that they are located in multiple locales. In optional embodiments, the databases include an oil characteristic database that includes data related to the characteristics (e.g., color, viscosity, smoke temperature, appropriate maintenance temperature for cooking, etc.) for multiple discrete types of cooking oil. Such information may be selectively transmitted to interactive assembly 110, for instance, as one or more oil characteristic signals.

Remote server 404 includes a network interface 520B such that interactive remote server 404 can connect to and communicate over one or more networks (e.g., network 502) with one or more network nodes. Network interface 520B can be an onboard component or it can be a separate, off board component. In turn, remote server 404 can exchange data with one or more nodes over the network 502. In particular, remote server 404 can exchange data with interactive assembly 110. It is understood that remote server 404 may further exchange data with any number of client devices over the network 502. The client devices can be any suitable type of computing device, such as a general-purpose computer, special purpose computer, laptop, desktop, integrated circuit, mobile device, smartphone, tablet, or other suitable computing device. In the case of a social media platform, images (e.g., static images or dynamic video), audio, or text may thus be exchanged between interactive assembly 110 and various separate client devices through remote server 404.

In optional embodiments, cooking appliance 300 is in operable communication with interactive assembly 110 (or a portion thereof, such as one or more camera assemblies 114A, 114B) via network 502. In turn, controller 510C of cooking appliance 300 may exchange signals with interactive assembly 110. Optionally, one or more portions of cooking appliance 300 may be controlled according to signals received from controller 510A of interactive assembly 110. For instance, one or more heating elements 326, 332 of cooking appliance 300 may be activated or directed to a specific heat output [e.g., in units of British Thermal Units, temperature (such as degrees Celsius or Fahrenheit), or relative heat settings (e.g., high, medium, low, etc.) based on one or more instruction signals received from controller 510A of interactive assembly 110 or remote server 404 (e.g., based on a type of identified cooking oil).

In certain embodiments, a user device 408 is communicatively coupled with network 502 such that user device 408 can communicate with interactive assembly 110. User device 408 can communicate directly with interactive assembly 110 via network 502. Alternatively, user 402 can communicate indirectly with interactive assembly 110 by communicating via network 502 with remote server 404, which in turn communicates with interactive assembly 110 via network 502. Moreover, user 402 can be in operative communication with user device 408 such that user 402 can communicate with interactive assembly 110 via user device 408.

User device 408 can be any type of device, such as, for example, a personal computing device (e.g., laptop or desktop), a mobile computing device (e.g., smartphone or tablet), a gaming console or controller, a wearable computing device, an embedded computing device, a remote, or any other suitable type of user computing device. User device 408 can include one or more user device controllers 510E. Controller 510E can include one or more processors 512E and one or more memory devices 514E. The one or more processors 512E can be any suitable processing device (e.g., a processor core, a microprocessor, an ASIC, a FPGA, a controller, a microcontroller, etc.) and can be one processor or a plurality of processors that are operatively connected. The memory device (i.e., memory) can include one or more non-transitory computer-readable storage mediums, such as RAM, ROM, EEPROM, EPROM, flash memory devices, magnetic disks, etc., and combinations thereof. The memory can store data and instructions which are executed by the processor 512E to cause user device 408 to perform operations. Controller 510E may include a user device network interface 520E such that user device 408 can connect to and communicate over one or more networks (e.g., network 502) with one or more network nodes. Network interface 520E can be an onboard component of controller 510E or it can be a separate, off board component. Controller 510E can also include one or more transmitting, receiving, or transceiving components for transmitting/receiving communications with other devices communicatively coupled with user device 408. Additionally or alternatively, one or more transmitting, receiving, or transceiving components can be located off board controller 510E.

User device 408 can include one or more user inputs 418 (e.g., buttons, knobs, one or more cameras, etc.) or a monitor 420 configured to display graphical user interfaces or other visual representations to user. For example, monitor 420 can display graphical user interfaces corresponding to operational features of interactive assembly 110 such that user may manipulate or select the features to operate interactive assembly 110. Monitor 420 can be a touch sensitive component (e.g., a touch-sensitive display screen or a touch pad) that is sensitive to the touch of a user input object (e.g., a finger or a stylus). For example, a user 402 may touch the monitor 420 with his or her finger and type in a series of numbers on the monitor 420. In addition, motion of the user input object relative to the monitor 420 can enable user 402 to provide input to user device 408. User device 408 may provide other suitable methods for providing input to user device 408 as well. Moreover, user device 408 can include one or more speakers, one or more cameras, or more than one microphones such that user device 408 is configured with voice control, motion detection, and other functionality.

Generally, user 402 may be in operative communication with interactive assembly 110, cooking appliance 300, or one or more user devices 408. In some exemplary embodiments, user 402 can communicate with devices (e.g., interactive assembly 110) using voice control. User 402 may also be in operative communication via other methods as well, such as visual communication.

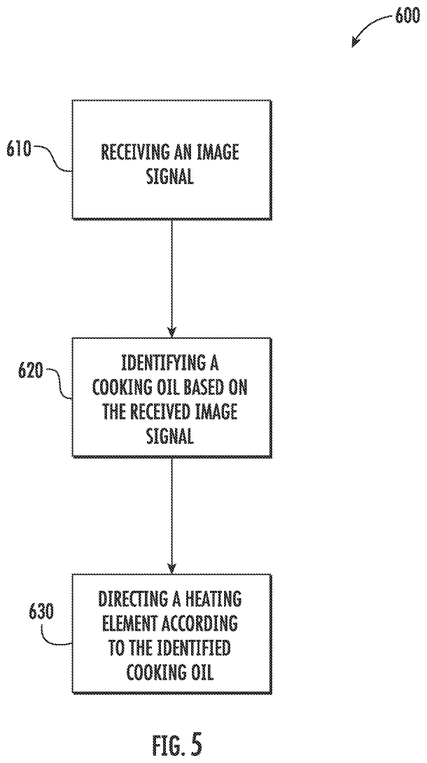

Referring now to FIG. 5, various methods may be provided for use with system 100 (FIG. 1) in accordance with the present disclosure. In general, the various steps of methods as disclosed herein may, in exemplary embodiments, be performed by the controller 510A (FIG. 4) as part of an operation that the controller 510A is configured to initiate (e.g., a directed cooking operation). During such methods, controller 510A may receive inputs and transmit outputs from various other components of the system 100. For example, controller 510A may send signals to and receive signals from remote server 404, cooking appliance 300, or user device 408, as well as other components within interactive assembly 110 (FIG. 4). In particular, the present disclosure is further directed to methods, as indicated by 600, for operating system 100. Such methods advantageously facilitate adaptive cooking that is responsive to the type of cooking oil being used without requiring direct user input or knowledge of the characteristics the particular cooking oil being used

FIG. 5 depicts steps performed in a particular order for purpose of illustration and discussion. Those of ordinary skill in the art, using the disclosures provided herein, will understand that (except as otherwise indicated) the steps of any of the methods disclosed herein can be modified, adapted, rearranged, omitted, or expanded in various ways without deviating from the scope of the present disclosure.

At 610, the method 600 includes receiving an image signal from a camera assembly. For instance, the image signal may be received from a camera assembly directed at (or otherwise adjacent to) the cooking zone of the cooking appliance, such as the first or second camera assembly of an interactive assembly. Optionally, multiple images (e.g., the first and second images) may be captured at the same camera assembly or, alternatively, at another camera assembly.

As described above, the camera assembly may be positioned directly above the cooking zone or cooktop surface of the cooking appliance. Moreover, the camera assembly may be directed toward the cooking zone (e.g., to capture portion of the cooktop surface that includes one or more individual heating elements, which may receive the cookware item thereon). Thus, the image signal may generally correspond to portion of the cooktop surface. As would be understood, the image signal may include multiple sequenced images captured by the camera assembly.

Generally, the image signal may be received in response to an image capture sequence initiated at the camera assembly or interactive assembly, as described above. In some embodiments, the image signal may be captured and transmitted by specific user input supplied to a control panel or image monitor (e.g., touchscreen) of the interactive assembly. During the image capture sequence, an image may be captured that includes a particular cooking oil (e.g., within a cookware item or storage vessel). The image may then be included with the image signal received at 610 (e.g., for further analysis at the interactive assembly, remote server, etc.).

Optionally, one or more heating elements within the cooking zone may be activated subsequent or, alternatively, prior to 610. Once activated, a temperature may be detected at or adjacent to the heating element(s) of the cooking zone. In particular, the temperature may be detected using the camera assembly (e.g., simultaneously with or separate from 610) or, alternatively, using a separate temperature sensor mounted to the cooking zone.

At 620, the method 600 includes identifying a cooking oil based on the received image signal. In particular, the type of cooking oil captured within the image of 610 may be identified. Identification may be performed, at least in part, by any suitable routine or sequence applied to the received image signal, such as edge matching, divide-and-conquer search, greyscale matching, histograms of receptive field responses, etc.

As an example, as described above, the cooking oil may be detected from a storage vessel captured within the image. As an additional or alternative example, and as also described above, the cooking oil may be detected as a liquid volume within a cookware item based on the received image signal. Optionally, detection of the cooking oil as a liquid may be first established before further analysis and precise identification of the type of cooking oil. Additionally or alternatively, a smoke presence may be detected. In other words, it may be determined that smoke from the cooking oil is captured within the image. Separate from or in addition to smoke presence, a temperature at the cooking zone may be detected (e.g., from the camera assembly or a separate temperature sensor, as would be understood). Thus, it may be determined at what temperature the volume of cooking oil within a cookware item begins to emit smoke.

Along with basic identifying information (e.g., a common name) of the cooking oil, one or more characteristics of the cooking oil may be determined at 620. For instance, a database or chart may be provided that includes or correlates one or more characteristics of multiple discrete cooking oils. Such characteristics may include a maintenance temperature associated with the cooking oil. Specifically, the maintenance temperature may indicate a temperature value or range of values at which the associated oil may be heated without emitting smoke or imparting an undesirable flavor to one or more food items. Such characteristics may also (i.e., additionally or alternatively) include a smoke temperature associated with the cooking oil, as described above.

Once identified, information concerning the cooking oil (e.g., identifying information, oil characteristics, etc.) may be displayed as a notification. In particular, the notification may be displayed at a control panel of the cooking appliance, at the interactive assembly, or a remote user device.

At 630, the method 600 includes directing the heating element according to the identified cooking oil. Thus, the activation of the heating element may be controlled based on the type of cooking oil captured at 610. If the cooking oil is identified over a particular heating element of a plurality of heating elements, 630 may correspond to that same heating element. In some embodiments, the heat output at the heating element is adjusted (e.g., increased or decreased) based on one or more stored characteristics of the identified cooking oil. For instance, the heating element may be controlled on a closed loop (e.g., in cooperation with an IR or temperature sensor) to maintain a temperature that is at (i.e., equal to) a maintenance temperature (e.g., within a prescribed range) or below a smoke temperature. If the temperature at the heating element is detected as being greater than or equal to a smoke temperature, activation of the heating element may be halted (e.g., for a predetermined period of time or until a new temperature condition is met).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A cooking assembly comprising:
a cooking appliance comprising a heating element defining a cooking zone selectively heated by the heating element;
a camera assembly directed at the cooking zone to capture one or more images thereof; and
a controller in operable communication with the heating element and the camera assembly, the controller being configured to initiate a cooking operation comprising
receiving an image signal from a camera assembly adjacent to the cooking zone,
identifying a cooking oil based on the received image signal, and
directing the heating element according to the identified cooking oil.

2. The cooking assembly of claim 1, wherein identifying the cooking oil comprises determining a maintenance temperature associated with the cooking oil, and wherein directing the heating element comprises directing the heating element to the maintenance temperature.

3. The cooking assembly of claim 1, wherein identifying the cooking oil comprises detecting a smoke presence based on the received image signal.

4. The cooking assembly of claim 1, wherein identifying the cooking oil comprises detecting a liquid volume within a cookware item based on the received image signal.

5. The cooking assembly of claim 1, wherein identifying the cooking oil comprises detecting a storage vessel based on the received image signal.

6. The cooking assembly of claim 1, wherein directing the heating element comprises adjusting heat output at the heating element based on one or more stored characteristics of the identified cooking oil.

7. The cooking assembly of claim 1, wherein the cooking operation further comprises detecting a temperature at the cooking zone in tandem with the image signal.

8. The cooking assembly of claim 7, wherein identifying the cooking oil comprises determining a smoke temperature associated with the cooking oil, wherein detecting the temperature at the cooking zone comprises determining the temperature at the cooking zone is greater than or equal to the smoke temperature, and wherein directing the heating element comprises halting activation of the heating element in response to determining the temperature at the cooking zone is greater than or equal to the smoke temperature.

9. The cooking assembly of claim 1, wherein the cooking operation further comprises initiating a notification of the identified cooking oil at a control panel of the cooking appliance.

10. A method of operating a cooking appliance comprising a heating element and defining a cooking zone selectively heated by the heating element, the method comprising:
receiving an image signal from a camera assembly adjacent to the cooking zone;
identifying a cooking oil based on the received image signal; and
directing the heating element according to the identified cooking oil.

11. The method of claim 10, wherein identifying the cooking oil comprises determining a maintenance temperature associated with the cooking oil, and wherein directing the heating element comprises directing the heating element to the maintenance temperature.

12. The method of claim 10, wherein identifying the cooking oil comprises detecting a smoke presence based on the received image signal.

13. The method of claim 10, wherein identifying the cooking oil comprises detecting a liquid volume within a cookware item based on the received image signal.

14. The method of claim 10, wherein identifying the cooking oil comprises detecting a storage vessel based on the received image signal.

15. The method of claim 10, wherein directing the heating element comprises adjusting heat output at the heating element based on one or more stored characteristics of the identified cooking oil.

16. The method of claim 10, further comprising:
detecting a temperature at the cooking zone in tandem with the image signal.

17. The method of claim 16, wherein identifying the cooking oil comprises determining a smoke temperature associated with the cooking oil, wherein detecting the temperature at the cooking zone comprises determining the temperature at the cooking zone is greater than or equal to the smoke temperature, and wherein directing the heating element comprises halting activation of the heating element in response to determining the temperature at the cooking zone is greater than or equal to the smoke temperature.

18. The method of claim 10, further comprising
initiating a notification of the identified cooking oil at a control panel of the cooking appliance.
